(19) United States
(12) Patent Application Publication
(10) Pub. No.: US 2005/0258402 A1
(43) Pub. Date: Nov. 24, 2005

(54) Odorant for gas

(76) Inventors: Michael Haubs, Bad Hreuznach (DE); Kai-Uwe Clauswitz, Gladenbach (DE)

Correspondence Address: CONNOLLY BOVE LODGE & HUTZ, LLP, P O BOX 2207, WILMINGTON, DE, 19899, US

(21) Appl. No.: 10/433,240

(22) PCT Filed: Oct. 29, 2001
(86) PCT No.: PCT/EP01/12472

§ 371 Date: Oct. 10, 2003

(30) Foreign Application Priority Data
Nov. 27, 2000 (DE) 100 58 805.0

(51) Int. Cl. G01N 33/22

(52) U.S. Cl. 252/408.1; 252/68

(57) ABSTRACT

The present invention relates to an odorant for town gas, comprising norbornene or a norbornene derivative with a diluent and optionally further additives. The odorant according to the invention is characterized in that it is sulfur-free and of low toxicity, but is absorbed by the earth only in a small amount and develops a distinct warning odor even in low concentrations.

Natural gas for the public gas supply is odorized worldwide without exception using sulfur-containing odorants. For example, mercaptans or thiophenes are used. These substances are added in order to give a warning odor to natural gas, which is itself virtually odorless. Although the substances used today are highly suitable for the odorization, their combustion releases sulfur dioxide, which pollutes the environment. In addition, there is interest in the localized use of odorized natural gas for catalytic processes, for example use in fuel cells. In order not to reduce the activity of the catalysts used, it is of interest to have the smallest possible amounts of sulfur compounds which act as catalyst poisons in the natural gas. For this reason, sulfur-free odorants have already been investigated. In JP 55056190, 5-ethylidene-2-norbornene and 5-vinyl-2-norbornene are used as sulfur-free odorants, in JP 08060167 together with pyrazine derivatives. DE 198 37 066 A1 describes the use of acrylic esters as sulfur-free odorant. A disadvantage here is, in particular, the absorption tendency of the odorant in the soil, the polymerization tendency of the acrylic esters and the inadequate volatility of the norbornene derivatives used and of the proposed mixtures. Odorants have to satisfy the basic requirements specified in DVGW Arbeitsblatt [Worksheet] G280 (DVGW—Deutscher Verein des Gas- und Wasserfaches e.V. [The German Technical and Scientific Association for Gas and Water], Eschborn). Specifically, these are:
warning odor intensity
warning odor character
toxicity
harmful components in waste gas
vaporization behavior
stability and
detectability.

Moreover, it is desired to achieve a low adsorption tendency of the odorant to pipe walls or in the soil. If there are leakages in the underground pipe system, the natural gas permeates initially layers of earth, in which odorants can be adsorbed. The natural gas then emerges odorless, which has led to serious accidents. A further important safety criterion is therefore the adsorption behavior of the odorant in the soil.

The object of the present invention was to find an odorant for town gas which satisfies the basic requirements given above and has a low adsorption tendency of the odorant to pipe walls and in the soil.

The object is achieved by a mixture of at least two components, where one of the components is norbornene or a norbornene derivative, and one component is a diluent. In an advantageous embodiment of the present invention, the odorant for gas comprises norbornene or a norbornene derivative, a diluent and further substance.

Surprisingly, it has been found that the mixtures according to the invention satisfy all of the requirements. In particular, the novel odorant has excellent adsorption properties due to the nonpolar character of the norbornene.

Pure norbornene is less suitable as an odorant since it is solid at room temperature (melting point: 45° C.). An important requirement for odorants, however, is that the cloud point is at a temperature of −30° C. or below. The cloud point is the temperature at which a liquid odorant clouding can be visually observed as a result of solidification of the melt or of the precipitation of one component. The diluent thus lowers the cloud point of the norbornene or of the norbornene derivative. Mixtures of norbornene or norbornene derivatives with diluents satisfy all of the requirements. The norbornene derivatives which can be used according to the invention are generally compounds which comprise a norbornene or norbornane structure, but where the basic structure of the bicycle must not contain any heteroatoms. Advantageously, it is possible to use norbornene derivatives, such as norbornenecarboxylic acids, alcohols derived from norbornene, but in particular nonpolar norbornene derivatives. These norbornene derivatives can be norbornene or norbornadiene, its derivatives, norbornene and norbornadiene derivatives substituted by alkyl or aryl groups, in particular norbornene derivatives which can be prepared by Diels-Alder reaction of cyclopentadiene or cyclopentadiene derivatives, in particular pentamethylcyclopentadiene and unsaturated hydrocarbons, such as, for example, 1,2,3,4,7-pentamethylnorbornene, 5-ethylidene-2-norbornene and 5-vinyl-2-norbornene. Norbornene derivatives which may be used are also saturated compounds which contain a norbornane structure, such as, for example, camphane, bornane and also fenchane terpenes, and also trinorbornane and compounds derived therefrom. The suitable norbornene derivatives are generally known and are described, for example, in: Beilstein E IV 5: 394; Blechert, Nachrichen aus Chemie, Technik und Laboratorium, 28 (1980) 724-726; Buchbauer, Popp, Chemiker-Zeitung 107 (1983) 327-339; Burkert, Angew. Chem. 93 (1981) 602 f.; Kirk-Othmer (3.) 18: 436-442; Marchand, Stereochemical Applications of NMR Studies in Rigid Bicyclic Systems, Weinheim verl. Chemie 1982; Ohn, Chem-Tech 10 (1980) No. 3, p.183; Stein, Inform. Chimie 1982, No. 225, p. 179-182, to which reference is made.

The diluent should have an evaporation behavior similar to that or norbornene; for this reason, compounds which form azeotropes or azeotrope-like compositions with norbornene are particularly suitable. Suitable diluents are, in particular, aliphatic and aromatic hydrocarbons, such as petroleum ether, benzine, light benzine, methane, ethane, propane, butane, isobutane, and also linear and branched hydrocarbons having 5 to 10 carbon atoms, and also benzene, toluene, xylenes, ethylbenzene and diethylbenzene, and generally substituted benzene derivatives, in particular benzene derivatives which are substituted by alkyl radicals which contain 1 to 4 carbon atoms. Also suitable are esters, in particular formic esters, acetic esters, propionic esters, butanoic esters and valeric esters, in particular the esters with short-chain aliphatic alcohols, such as methanol, ethanol, propanol or butanol, such as, for example, methyl formate and ethyl formate, and also ethyl acetate. Linear and cyclic ethers are also suitable, such as, for example, tetrahydrofuran, dioxane and isopropyl methyl ether. Preferred diluents for use with norbornene are ethyl acetate and ethyl propionate, particular preference being given to aliphatic ethers, in particular tetrahydrofuran, very particular preference being given to hydrocarbons, such as aromatic hydrocarbons, toluene and xylene, in particular aliphatic hydrocarbons, of which particular preference is given to pentane, hexane, heptane, and isomers thereof, and also petroleum ether. Petroleum ether is the term generally used for a mixture of various, low-boiling (about 50 to 70° C.) hydrocarbons. The diluents can be used individually or as a mixture. The diluents must be at least partially miscible with norbornene and have a boiling point at atmospheric pressure of from 20° C. to 200° C. The diluents are used in a concentration range from 10% by weight to 80% by weight, preferably from 20 to 50% by weight. In an advantageous embodiment of the present invention, norbornene is soluble in the diluent and the odorant according to the invention is a solution of norbornene in the diluent. The cloud points of the mixtures according to the invention are at least −30° C., advantageously −40° C., in particular −41 ° C. or lower temperatures. Where appropriate, further substances can also be added in order to improve the warning odor character or in order to act as stabilizers, such as antioxidants. The concentration range of the further additives is in the range from 0.01 to 10% by weight, preferably in the range from 0.1 to 5% by weight. Further preferred embodiments are given in the claims.

### EXAMPLES

The cloud points of the examples are −30° C., unless stated otherwise.

### Example 1

| | |
|---|---|
| Norbornene: | 60 percent by weight |
| Toluene: | 40 percent by weight |

### Example 2

| | |
|---|---|
| Norbornene: | 65 percent by weight |
| Petroleum ether (boiling range 80-100° C.): | 35 percent by weight |

### Example 3

| | |
|---|---|
| Norbornene: | 70% by weight |
| Petroleum ether (boiling range 50-70° C.): | 30% by weight |
| Cloud point: | −41° C. |

This application is a 371 application of PCT/EP01/12472 filed Oct. 29, 2001 claims benefit to german application 100 58 805.0 filed Nov. 27, 2000.

1-16. (canceled)

17. An odorant for gas, comprising a mixture of at least two components, wherein one of the components is norbornene or a norbornene derivative, and one component is a diluent.

18. The odorant for gas as claimed in claim 17, which further comprises a further substance.

19. The odorant as claimed in claim 18, wherein the further substance is a substance for changing the warning odor character, a stabilizer, an antioxidant or a mixture thereof.

20. The odorant as claimed in claim 18, wherein the further substance is present in an amount of from 0.01 to 10% by weight.

21. The odorant as claimed in claim 18, wherein the further substance is present in an amount of from 0.1 to 5% by weight.

22. The odorant as claimed in claim 17, wherein the norbornene derivative used is one or more of the following compounds: norbornene, norbornenecarboxylic acid, norbornene dicarboxylic acid, alcohols derived from norbornene, norbornadiene, norbornene derivatives substituted by alkyl or aryl groups, norbornadiene derivatives substituted by alkyl or aryl groups, norbornane derivatives substituted by alkyl or aryl groups, norbornene derivatives which are prepared by Diels-Alder reaction of cyclopentadiene derivatives, and unsaturated hydrocarbons, 1,2,3,4,7-pentamethylnorbornene, camphane terpenes, bornane terpenes, fenchane terpenes, trinorbornane and compounds derived therefrom.

23. The odorant as claimed in claim 17, wherein the norbornene derivative used is cyclopentadiene or pentamethylcyclopentadiene

24. The odorant as claimed in claim 17, wherein the diluent used is a hydrocarbon, ether, ester or a mixture of these substances with a boiling point or boiling range between 20 and 200° C.

25. The odorant as claimed in claim 17, wherein the diluent used is one or more of the following compounds: aliphatic hydrocarbons, aromatic hydrocarbons, petroleum ether, benzine, light benzine, methane, ethane, propane, butane, isobutane, linear hydrocarbons having 5 to 10 carbon atoms, branched hydrocarbons having 5 to 10 carbon atoms, also benzene, toluene, xylenes, ethylbenzene, diethylbenzene, benzene derivatives which are substituted by alkyl radicals which contain 1 to 4 carbon atoms, esters, formic esters, acetic esters, propionic esters, butanoic esters, valeric esters, esters of a carboxylic acid with short-chain aliphatic alcohols, such as methanol, ethanol, propanol or butanol, methyl formate and ethyl formate, ethyl acetate, linear ethers, cyclic ethers, tetrahydrofuran, dioxane isobutyl methyl ether.

26. The odorant as claimed in claim 22, wherein the diluent used is one or more of the following compounds: aliphatic hydrocarbons, aromatic hydrocarbons, petroleum ether, benzine, light benzine, methane, ethane, propane, butane, isobutane, linear hydrocarbons having 5 to 10 carbon atoms, branched hydrocarbons having 5 to 10 carbon atoms, also benzene, toluene, xylenes, ethylbenzene, diethylbenzene, benzene derivatives which are substituted by alkyl radicals which contain 1 to 4 carbon atoms, esters, formic esters, acetic esters, propionic esters, butanoic esters, valeric esters, esters of a carboxylic acid with short-chain aliphatic alcohols, such as methanol, ethanol, propanol or butanol, methyl formate and ethyl formate, ethyl acetate, linear ethers, cyclic ethers, tetrahydrofuran, dioxane isobutyl methyl ether.

27. The odorant as claimed in claim 17, wherein the mixture contains 10 to 80% by weight of diluent.

28. The odorant as claimed in claim 17, wherein the mixture contains 20 to 50% by weight of diluent.

29. The odorant as claimed in claim 17, wherein the mixture contains 10 to 80% by weight of norbornene or a norbornene derivative.

30. The odorant as claimed in claim 17, wherein the mixture contains less than 10% by weight of norbornene or a norbornene derivative.
